(19) United States
(12) Patent Application Publication
(10) Pub. No.: US 2025/0195499 A1
(43) Pub. Date: Jun. 19, 2025

(54) METHODS FOR PREVENTING OR TREATING CEREBROVASCULAR DISEASES OR TISSUE ISCHEMIA-REPERFUSION INJURY

(71) Applicant: CO., LTD. OF JEAMOON TECHNOLOGY, Beijing (CN)

(72) Inventors: Qinghua CUI, Beijing (CN); Wanlu ZHOU, Beijing (CN)

(73) Assignee: CO., LTD. OF JEAMOON TECHNOLOGY, Beijing (CN)

(21) Appl. No.: 19/062,065

(22) Filed: Feb. 25, 2025

Related U.S. Application Data
(63) Continuation of application No. PCT/CN2023/110548, filed on Aug. 1, 2023.

(30) Foreign Application Priority Data
Aug. 26, 2022 (CN) 202211031566.1

(51) Int. Cl. A61K 31/4709; A61P 9/10

(52) U.S. Cl. CPC A61K 31/4709; A61P 9/10

(57) ABSTRACT

A method for preventing or treating a cerebrovascular disease or a tissue ischemia-reperfusion injury is provided. The method comprises administering to a patient a drug containing a pharmaceutically effective amount of LY2922470. The embodiments of the present disclosure confirm the significant potential of LY2922470 as a drug for cerebrovascular diseases and tissue ischemia-reperfusion injury. For the first time, it is disclosed that LY2922470 effectively improves infarct lesions, providing an effective new potential alternative drug for the treatment of related cerebrovascular diseases and tissue ischemia-reperfusion injury, expanding the indications of LY2922470, and greatly enhancing the application potential and market prospects of LY2922470.

### CROSS-REFERENCE TO RELATED APPLICATIONS

This application is a Continuation of International Application No. PCT/CN2023/110548, filed on Aug. 1, 2023, which claims priority to Chinese Patent Application No. 202211031566.1, filed on Aug. 26, 2022, the entire contents of each of which are hereby incorporated by reference.

### TECHNICAL FIELD

The present disclosure relates to the technical field of biomedicine, and in particular, to a method for preventing or treating cerebrovascular diseases or tissue ischemia-reperfusion injury.

### BACKGROUND

LY2922470, developed by Eli Lilly and Company, functions as a small molecule activator of GPR40 for the treatment of Type 2 Diabetes Mellitus (T2DM). Its chemical structure is as follows:

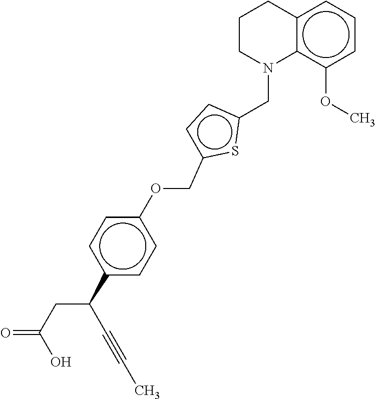

GPR40 is a member of the G protein-coupled receptor (GPCR) family. Activation of GPR40 has been demonstrated to induce glucose-stimulated insulin secretion (GSIS) in pancreatic B-cells and incretin secretion in enteroendocrine cells. Consequently, GPR40 has emerged as a viable and promising therapeutic target for T2DM, particularly due to its lack of hypoglycemia risk. This has garnered significant attention as a drug target for T2DM, leading to the development and investigation of numerous GPR40 ligands for their antidiabetic effects. Among the GPR40 ligands, TAK875, a GPR40 activator, has been successfully tested in Phase II clinical trials.

The general formula of the LY2922470 compound structure was first disclosed in WO2015105786A1, which also proposed the use of this compound for treating T2DM.

Cerebrovascular diseases are a group of disorders that occur in the brain's blood vessels, causing brain tissue damage due to disturbances in intracranial blood circulation. Stroke is the second leading cause of death and the third leading cause of disability worldwide. The proportion of deaths caused by stroke is projected to increase to 24.9% by 2030, with ischemic stroke accounting for approximately 80% of all stroke cases.

At present, there are relatively few treatment strategies for ischemic stroke, and the clinically common and effective treatment method is thrombolysis with tissue plasminogen activator (t-PA) to restore blood supply and reduce neuronal damage. However, the brain is a highly energy-demanding organ that is extremely sensitive to ischemia and hypoxia. Although restoring blood flow perfusion is the primary clinical treatment for stroke, it may lead to cerebral ischemia-reperfusion (I/R) injury, which is one of the primary factors causing severe disability and death in patients. Currently, preventing stroke and repairing damage after ischemia-reperfusion remains an urgent and unmet need, urgently requiring the development of new drugs.

### SUMMARY

One or more embodiments of the present disclosure provide a method for preventing or treating ischemic stroke, comprising: administering to a patient a drug containing a pharmaceutically effective amount of LY2922470, wherein a structural formula of the LY2922470 is:

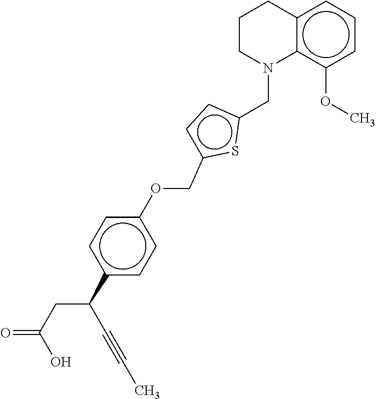

In some embodiments, the LY2922470 in the drug is in a form of a compound or a pharmaceutically acceptable salt thereof.

In some embodiments, the drug is in an oral or injectable dosage form, and the oral or injectable dosage form includes at least one of a powder, a tablet, a granule, a capsule, an oral liquid, an emulsion, and a suspension.

In some embodiments, the drug further contains an excipient.

### BRIEF DESCRIPTION OF THE DRAWINGS

The present disclosure will be further illustrated by way of exemplary embodiments, which will be described in detail through the accompanying drawings, wherein:

FIG. 1 illustrates laser speckle images of mice in a sham-operated control group according to some embodiments of the present disclosure;

FIG. 2 illustrates laser speckle images of mice subjected to middle cerebral artery occlusion (MCAO) model and ischemia-reperfusion according to some embodiments of the present disclosure;

FIG. 3 illustrates 2,3,5-triphenyltetrazolium chloride (TTC) staining results of brain tissue from each group of mice according to some embodiments of the present disclosure;

FIG. 4 is a diagram illustrating quantitative analysis results of infarct areas based on TTC staining according to some embodiments of the present disclosure; and

FIG. 5 is a schematic diagram illustrating an MCAO cerebral ischemia model using an intraluminal filament approach according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor, unless otherwise indicated or stated in the context, the same reference numerals in the drawings represent the same structures or operations.

Set forth in the present disclosure and the claims, unless explicitly indicated otherwise in the context, words such as “one”, “a”, “an”, and/or “the” do not specifically denote the singular form and may also include the plural form. In general, the terms “comprising” and “including” only suggest the inclusion of steps and elements that have been explicitly identified, and these steps and elements do not constitute an exclusive listing; methods may also include other steps or elements.

Unless otherwise defined, all technical and scientific terms used in the present disclosure have the same meaning as typically understood by those of ordinary skill in the art to which the present disclosure pertains.

One or more embodiments of the present disclosure provide a method for preventing or treating a cerebrovascular disease or a tissue ischemia-reperfusion injury, comprising: administering to a patient a drug containing a pharmaceutically effective amount of LY2922470, wherein a structural formula of the LY2922470 is:

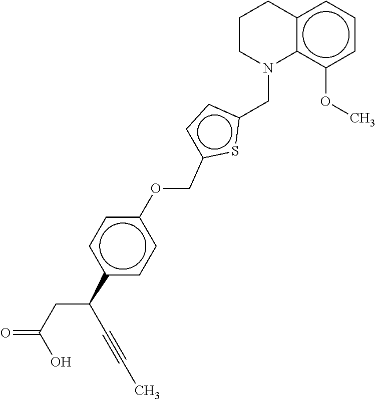

In some embodiments, the cerebrovascular disease includes ischemic stroke.

Ischemic stroke (also referred to as cerebral infarction or cerebral thrombosis) is an acute condition caused by impaired blood supply to the brain. A main pathological mechanism of the ischemic stroke involves the blockage or narrowing of cerebral blood vessels, leading to an interruption or reduction of the blood supply to local brain tissues, thus causing hypoxia and ischemic damage to the brain tissues.

In some embodiments, an ischemic stroke model may be constructed based on mice. Middle cerebral artery occlusion (MCAO) is a focal cerebral ischemia model and is currently one of the most widely used models of cerebral ischemia. Its pathogenesis is similar to that of human ischemic stroke, making it significant for creating models that simulate human cerebral ischemia and for studying the mechanisms of cerebral ischemia and drug screening. The embodiments of the present disclosure use the MCAO mouse model for pharmacodynamic verification.

In some embodiments, the drug contains an effective dose of LY2922470. The effective dose refers to an amount in a unit dose (e.g., the amount in one tablet, one injection, one pill, or one dose of medication) or an amount in a unit dose for a treated patient (e.g., a dose per unit of body weight). In some embodiments, subjects of the drug treatment include mammals, including humans, canines, rodents, etc. The conversion of effective doses between different animals may be based on an equivalent dose conversion relationship between experimental animals and humans in the field (typically referenced from guidelines by regulatory agencies such as the FDA or SFDA, or from literature such as “Huang Jihan et al. Equivalent Dose Conversion between Animals and Between Animals and Humans in Pharmacological Experiments. Chinese Journal of Clinical Pharmacology and Therapeutics, 2004 September; 9 (9): 1069-1072”). Thus, the unit body weight dose for humans may be extrapolated from the dose used in experimental animals. For example, for commonly used experimental animals such as mice, the conversion ratio to adult humans is approximately 12:1 according to the aforementioned literature.

In some embodiments, the effective dose (calculated by content) for treating ischemic stroke in 8-week-old C57BL/6J mice is 2.5-120 mg/kg. Preferably, the effective dose (calculated by content) for treating ischemic stroke in 8-week-old C57BL/6J mice is 10-80 mg/kg. Based on the conversion relationship between effective doses in mice and adults, with a standard adult body weight set at 60 kg, the effective dose for adults is 12.5-600 mg per day. Preferably, the effective dose for adults is 50-400 mg per day.

In some embodiments, the LY2922470 in the drug is in a form of a compound or a pharmaceutically acceptable salt thereof.

In some embodiments, the drug is in an oral or injectable dosage form, and the oral or injectable dosage form includes at least one of a powder, a tablet, a granule, a capsule, an oral liquid, an emulsion, and a suspension.

In some embodiments, the drug further contains an excipient or a cardiovascular and cerebrovascular drug.

In some embodiments, the drug further contains a pharmaceutically acceptable carrier. The carrier includes at least one of a diluent, a buffer, a suspending agent, an emulsion, a granule, an encapsulating agent, an excipient, a filler, a binder, a spray, a transdermal absorbent, a wetting agent, a disintegrant, an absorption enhancer, a surfactant, a colorant, a flavoring agent, and an adsorbent carrier.

In some embodiments of the present disclosure, a new indication for the LY2922470 drug is provided. As a drug for ischemic stroke, LY2922470 can effectively improve the infarct lesion, providing a new alternative drug for preventing and treating ischemic stroke and tissue ischemia-reperfusion injury.

One or more embodiments of the present disclosure further provide a use of LY2922470 in the preparation of a drug for preventing or treating a cerebrovascular disease or a tissue ischemia-reperfusion injury, wherein the structural formula of the LY2922470 is:

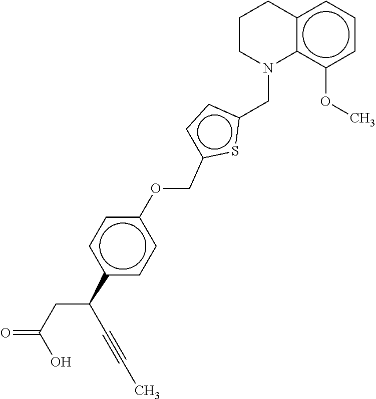

In some embodiments, the cerebrovascular disease includes ischemic stroke.

In some embodiments, the LY2922470 in the drug is in a form of a compound or a pharmaceutically acceptable salt thereof.

In some embodiments, the drug is in an oral or injectable dosage form, and the oral or injectable dosage form includes at least one of a powder, a tablet, a granule, a capsule, an oral liquid, an emulsion, and a suspension.

In some embodiments, the drug further contains an excipient or a cardiovascular and cerebrovascular drug.

In some embodiments, the drug further contains a pharmaceutically acceptable carrier. The carrier includes at least one of a diluent, a buffer, a suspending agent, an emulsion, a granule, an encapsulating agent, an excipient, a filler, a binder, a spray, a transdermal absorbent, a wetting agent, a disintegrant, an absorption enhancer, a surfactant, a colorant, a flavoring agent, and an adsorbent carrier.

The embodiments of the present disclosure have at least the following beneficial effects:

By using the MCAO cerebral ischemia-reperfusion injury animal model to evaluate the therapeutic effects of LY2922470 and TAK875 on ischemic stroke, the results show that LY2922470 has a therapeutic effect on ischemic stroke, and the therapeutic effect is not directly related to the GPR40 target. The embodiments of the present disclosure confirm the significant potential of LY2922470 as a drug for cerebrovascular diseases and ischemia-reperfusion injury. For the first time, it is disclosed that LY2922470 can effectively improve infarct lesions, providing an effective new potential alternative drug for the treatment of related cerebrovascular diseases and reperfusion injury, thus expanding the indications of LY2922470 and greatly improving the application potential and market prospects of LY2922470.

The following examples are more specific descriptions related to some of the above embodiments. Some parts of these examples can be replaced or combined with corresponding contents of other examples to form new examples. Unless otherwise specified, the experimental methods in the following examples are conventional methods. Unless otherwise specified, the test materials used in the following examples are obtained from conventional biochemical reagent companies. In the quantitative tests in the following examples, three repeated experiments are set up, and the results are taken as the average. It should be understood that the following examples are for better explanation of the embodiments of the present disclosure and are not intended to limit the embodiments of the present disclosure.

### EXAMPLES

### Example 1. Construction of a Middle Cerebral Artery Occlusion (MCAO) Mouse Model

### I. Experimental Materials

1. Experimental animals: 8-week-old C57BL/6J mice, SPF grade, male, provided by SPF (Beijing) Biotechnology Co., Ltd.

2. Experimental instruments: gas anesthesia machine, isoflurane, finished filament (CINONTECH 1220-A5), stereomicroscope (Olympus SZ61), laser speckle blood flow imaging system (Rayward RFLSI III), sterilized instrument set (micro tweezers, micro scissors, hemostats, needle holders).

### II. Method for Constructing the MCAO Model (Intraluminal Filament Approach)

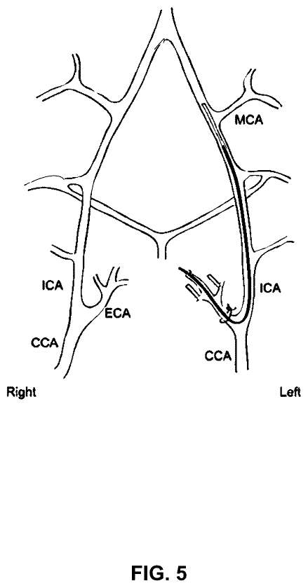

A surgical incision was made in the neck to expose a left common carotid artery (CCA), an internal carotid artery (ICA), and an external carotid artery (ECA). A pre-made filament was inserted through the external carotid artery into the internal carotid artery and advanced to a middle cerebral artery (MCA), causing ischemia in a left brain region. After 60 minutes of occlusion, the filament was removed to achieve reperfusion in the left brain region. FIG. 5 is a schematic diagram illustrating the MCAO cerebral ischemia model using the intraluminal filament approach.

### Surgical Procedure:

1. Animal anesthesia and neck preparation: the mice were anesthetized by inhalation of 2% isoflurane gas. After muscle relaxation, the four limbs of the mice were fixed to an operating table with 3M medical tape. Depilatory cream was used to remove the hair on a side of the neck, and erythromycin eye ointment was applied to the eyeballs of the mice to prevent eye dryness.

2. Surgical incision: the neck skin was disinfected sequentially with iodophor and 75% alcohol cotton balls. A 3 cm incision was made along the midline of the neck. Under a stereomicroscope, the subcutaneous tissue and fat were bluntly dissected, the superficial fascia and platysma were incised to expose the deep fascia of the neck and the anterior tracheal muscle. After the anterior tracheal muscle was exposed, the deep cervical fascia was torn along an anterior edge of the left sternocleidomastoideole, exposing the sternocleidomastoideole, which was then pulled posteriorly. The dissection continued downward along the sternocleidomastoideole until the deeper carotid sheath was exposed.

3. Vessel exposure and isolation: while avoiding compression of the mouse trachea, the carotid sheath was incised, and the common carotid artery (CCA) was isolated, taking care not to damage the vagus nerve running alongside in the sheath, as simulation or injury to the nerve may cause sudden respiratory arrest in the animal. The dissection continued upward along the common carotid artery, and the external carotid artery was identified at an upper edge of the thyroid cartilage. The external carotid artery is located medial and superior to the internal carotid artery, near the trachea. A proximal end of the external carotid artery was gently lifted to expose the internal carotid artery located laterally and inferiorly.

4. Blood flow blockade: the common carotid artery was isolated and temporarily blocked using a 6-0 silk ligature in a slipknot. The internal carotid artery was temporarily clamped with a microvascular clip. Further dissection along the external carotid artery reveals a second branch, a lingual artery, an occipital artery, and an external maxillary artery. The branch vessels were coagulated using an electrocautery pen. A distal end of the external carotid artery was permanently ligated with a 6-0 silk ligature, while the proximal end was looped for later use.

5. Filament insertion: a small incision was made at a ligated distal end of the external carotid artery, and the filament was threaded through the incision and tied with the pre-prepared loop ligature. The incision was cut off, and the filament was adjusted and threaded into the internal carotid artery. The filament was further advanced until slight resistance was felt, indicating that a tip of the filament has reached the middle cerebral artery. The ligature was tightened to fix the filament and timing started immediately.

6. After suturing the skin, anesthesia was discontinued. Following 60 minutes of ischemia, the filament was removed to complete cerebral ischemia-reperfusion.

7. Close the incision: the incision was closed using interrupted 4-0 sutures, disinfected with iodophor, and 3 mL of saline was injected intraperitoneally for rehydration. Postoperative analgesia jelly was provided, and the mice were kept warm and monitored continuously until they recover from anesthesia.

### III. Verification of the MCAO Mouse Model by a Small Animal Laser Speckle Blood Flow Imaging System

Five mice were randomly selected from a sham-operated control group and six mice were randomly selected from a MCAO model group. Cerebral hemisphere blood flow during the MCAO modeling process was detected using the small animal laser speckle blood flow imaging system. The hair on the heads of the mice was shaved using an electric razor, and the scalp was disinfected with iodine tincture, followed by deiodination with 75% alcohol to prevent skin damage. The head skin was dried with clean cotton balls. A scalpel was used to make an incision along the midline of the parietal bone, and surgical scissors were used to cut an 18 mm×18 mm scalp incision at the parietal bone location. The tissue above the parietal bone was gently removed to expose the skull. A laser speckle imaging device was positioned above the parietal bone, and a set of laser speckle images was captured to detect the cerebral blood flow supply in the hemispheres during the MCAO modeling process.

### IV. Experimental Results and Conclusions

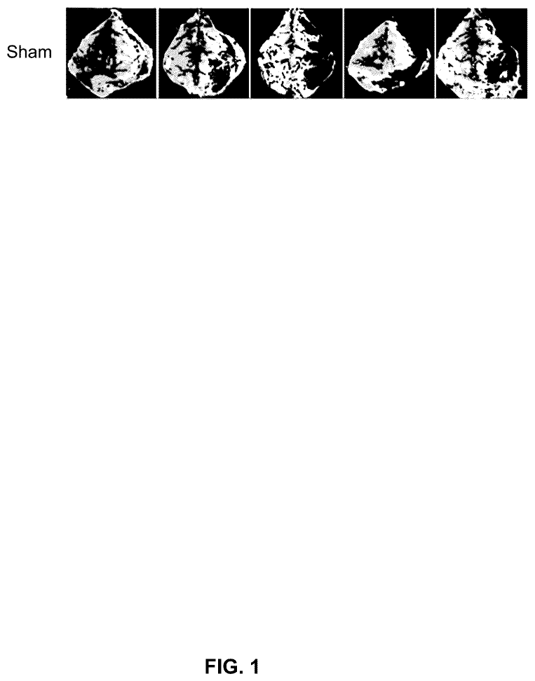

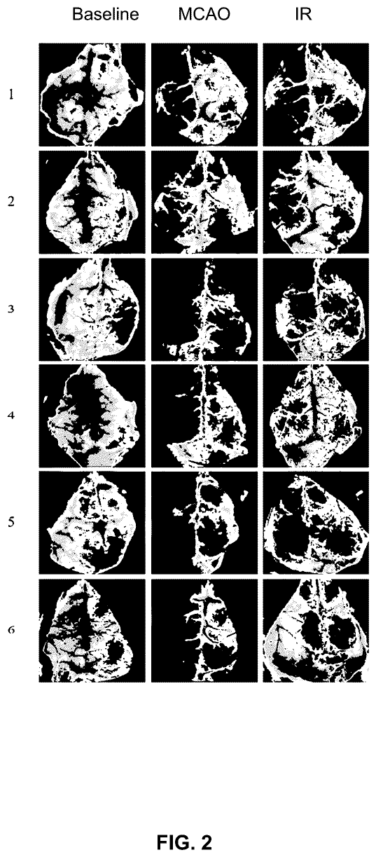

The Doppler laser speckle images of 5 mice from the sham-operated control group and 6 mice from the MCAO model group are shown in FIG. 1 and FIG. 2, respectively. The mice in the sham-operated control group exhibited normal cerebral blood flow. In contrast, the mice in the MCAO model group showed a significant reduction in blood flow in the left cerebral hemisphere after middle cerebral artery occlusion, with partial restoration of blood flow following reperfusion. The results are consistent with the pathophysiological characteristics of a MCAO cerebral ischemia-reperfusion (IR) model in mice, indicating the successful establishment of the ischemic stroke mouse model.

### Example 2: The Preventive and Therapeutic Effects of LY2922470 on Ischemic Stroke and Tissue Ischemia-Reperfusion

### I. Experimental Materials

1. Experimental animals: 8-week-old C57BL/6J mice, SPF grade, male, provided by SPF (Beijing) Biotechnology Co., Ltd.

2. Experimental drugs: LY2922470 (Shanghai Tao Shu Biotechnology Co., Ltd.), TAK875 (Shanghai Tao Shu Biotechnology Co., Ltd.), TTC (Sigma, T8877-50G).

1) Preparation of TAK875: 27 mg of TAK875 powder was precisely weighed using a precision balance, dissolved in 9 mL of 0.8% CMC-Na solution, and sonicated for 5 minutes to prepare a 3 mg/mL white suspension. The suspension was store at 4° C. for later use.

2) Preparation of LY2922470: 80 mg of LY2922470 yellow crystalline powder was added to 200 μl of DMSO solution to form a yellow transparent solution. Then the solution was added in a batch-wise manner to 10 ml of 0.8% CMC-Na solution. The mixture was heated and sonicated for 5 hours to prepare an 8 mg/ml LY2922470 white suspension. The suspension was store at 4° C. for later use.

3. Experimental instruments: gas anesthesia machine, isoflurane, finished filament (CINONTECH 1220-A5), stereomicroscope (Olympus SZ61), sterilized instrument set (micro tweezers, micro scissors, hemostats, needle holders).

### II. Experimental Groups

a) Sham group: control group, with all procedures identical to the model group except no filament insertion.

b) IR model group: filament approach to induce MCAO.

c) LY-10 mg/kg group: IR mice+LY2922470 (10 mg/kg, po, QD).

d) LY-20 mg/kg group: IR mice+LY2922470 (20 mg/kg, po, QD).

e) LY-40 mg/kg group: IR mice+LY2922470 (40 mg/kg, po, QD).

f) LY-80 mg/kg group: IR mice+LY2922470 (80 mg/kg, po, QD).

g) TAK875 group: IR mice+TAK875 (30 mg/kg, po, QD).

Note: po: oral administration, QD: once daily.

### III. Experimental Procedures

C57BL/6J male mice, aged 8 weeks, were acclimatized for 1 week and then randomly assigned to 7 groups. Except for eight mice in the blank control group (i.e., the sham-operated control group), the remaining mice underwent MCAO surgery to induce cerebral ischemia and were randomly divided into 6 groups, including an IR model group, a TAK875 group (12 mice per group), and four LY2922470 groups with different concentrations (8 mice per group).

Except for the eight mice in the blank control group (i.e., the sham-operated control group), the remaining mice began receiving drug administration 4 days before surgery. TAK875 was administered at a dose of 30 mg/kg, while LY2922470 was administered at doses of 10 mg/kg, 20 mg/kg, 40 mg/kg, and 80 mg/kg, respectively. The sham-operated control group and the IR model group received only a solvent treatment, with an administration volume of 10 mL/kg, and all drugs were administered via oral gavage. On the 5th day of administration, an MCAO cerebral ischemia-reperfusion (IR) in the mice was prepared using the filament approach. After 1 hour of ischemia, reperfusion was performed. After twenty-four hours of reperfusion, brain tissues were collected for TTC staining, and the results were analyzed to confirm drug efficacy.

IV. Experimental methods

### 1. TTC Staining in Mice

Mice were intraperitoneally injected with a 10% chloral hydrate solution. After cardiac perfusion with saline, the brain was rapidly removed, ensuring the integrity of the brain tissue during extraction. The brain was immediately frozen in a −20° C. freezer for 20 minutes. Brain slices (6-7 slices) were cut at 2 mm intervals, with the first cut made at the midpoint of the line connecting the anterior pole of the brain and the optic chiasm; the second cut at the optic chiasm; the third cut at the level of the infundibulum; and the fourth cut between the infundibulum and the posterior tail of the hypothalamus (reference: Zhang Juntian, Editor. Modern Pharmacological Experimental Methods). The brain slices were immersed in 2% 2, 3, 5-triphenyltetrazolium chloride (TTC) solution and incubated at 37° C. in a light-protected water bath for 10 minutes. The container was gently shaken every 5 minutes to ensure thorough staining. After staining, the slices were photographed. Red areas were identified as normal brain tissues, while grayish-white areas were identified as infarcted tissues, as observed by the naked eye. After scanning, an area of the normal brain tissues and an area of the infarcted tissues was calculated using Image-Pro Plus image analysis software. An infarct area percentage for each group was calculated by dividing the area of the infarcted tissues by the area of the normal tissues.

### 2. Data Collection and Statistical Analysis

Statistical analysis was performed using GraphPad Prism 8.0 software.

Measurement data for each group were expressed as mean±standard deviation (mean±SD), and comparisons between multiple groups were analyzed using one-way ANOVA. Comparisons between two groups were analyzed using a t-test, with a p-value less than 0.05 considered to be statistically significant. For categorical data, comparisons between groups were analyzed using the chi-square test, with a p-value less than 0.05 considered to be statistically significant.

The remaining experimental methods were the same as in Example 1.

### V. Experimental Results and Conclusions

### 1. General Animal Conditions

Mice were administered drugs for 5 days before the MCAO cerebral ischemia surgery. No deaths occurred in any of the groups before administration, and the animals were in good condition. After the MCAO cerebral ischemia surgery was performed, the mice exhibited typical ischemic symptoms, including circling toward the ischemic side while crawling or being unable to bear weight on the ischemic side, with tipping toward the ischemic side. These symptoms are consistent with MCAO cerebral ischemia symptoms. One mouse from the IR model group and one from the TAK875 group died; the IR model group mouse died 10 hours after reperfusion, while the TAK875 group mouse died 18 hours after reperfusion. No other deaths were observed in the remaining mice.

### 2. TTC Staining

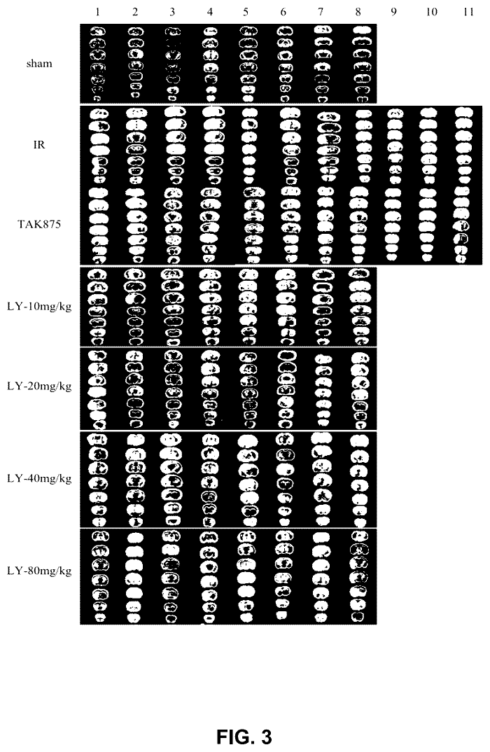

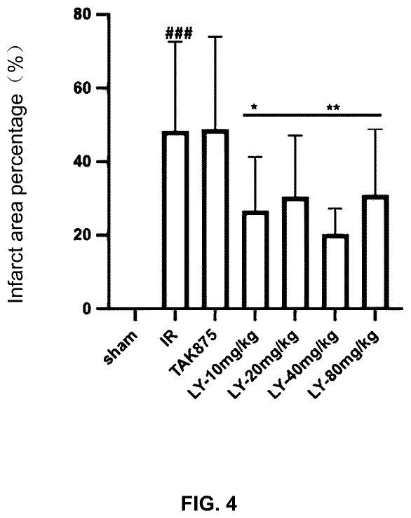

TTC staining was used to observe the cerebral infarct lesion, and the TTC staining results of brain tissue from each group of mice are shown in FIG. 3 (sham group, TAK875 group, n=11; other groups, n=8). FIG. 4 shows the quantitative analysis results of infarct areas by TTC staining (compared with the sham group: #P<0.05, ##P<0.01; compared with the IR group: * P<0.05, ** P<0.01), and infarcted area percentages are shown in Tab. 1.

As shown in FIG. 3 and FIG. 4, compared with the sham-operated control group mice, the infarcted area in the IR group mice was significantly larger. Compared with the infarcted area of the IR group mice, no significant decrease in the size of the infarcted area was observed after oral administration of the GPR40 agonist TAK875. After oral administration of different doses of LY2922470, the infarcted area in the mice was reduced. Compared with the IR group mice, the infarcted area in the brain of the mice treated with 10 mg/kg and 40 mg/kg of LY2922470 was significantly reduced. The results show that LY2922470 has a therapeutic effect on stroke and ischemia-reperfusion injury, and the therapeutic effect is not directly related to the GPR40 target.

Tab. 1 shows the pharmacodynamic statistics for LY2922470 on infarct area. The infarct area percentage in the IR model group was 48.42%. Compared with the IR model group, the infarct area in the LY2922470 groups significantly decreased, and the differences were statistically significant (NS: no statistical difference, *p<0.5, ** p<0.01, *** p<0.001). The data from the table show that LY2922470 demonstrated therapeutic effects at doses ranging from 10 mg/kg/day to 80 mg/kg/day, with the best therapeutic effect observed at doses of 10 mg/kg/day to 40 mg/kg/day. However, a protective effect of LY2922470 at doses below 10 mg/kg/day (e.g., 2.5-10 mg/kg) for brain tissue cannot be ruled out, nor can the potential therapeutic effect at doses higher than 80 mg/kg/day (e.g., 80-160 mg/kg) for stroke treatment.

TABLE 1

| Group | Infarct area percentage (%) X ± SD |
|---|---|
| Sham | 0 ± 0 |
| IR | 48.42 ± 24.21 (###) |
| TAK875 | 48.9 ± 25.13 |
| LY-10 mg/kg | 26.77 ± 14.54 (*) |
| LY-20 mg/kg | 30.55 ± 16.52 |
| LY-40 mg/kg | 20.4 ± 6.88 (**) |
| LY-80 mg/kg | 31.03 ± 17.8 |

The example of the present disclosure evaluated the therapeutic effects of LY2922470 and TAK875 on ischemic stroke using the MCAO cerebral ischemia-reperfusion injury animal model. The results showed that LY2922470 has a therapeutic effect on ischemic stroke, and the therapeutic effect is not directly related to the GPR40 target. Therefore, LY2922470 shows potential clinical application value in the treatment of ischemic stroke.

The basic concepts have been described above, apparently, in detail, as will be described above, and do not constitute limitations of the disclosure. Although there is no clear explanation here, those skilled in the art may make various modifications, improvements, and corrections of the present disclosure. This type of modifications, improvements, and corrections are recommended in the present disclosure, so the modifications, improvements, and the corrections remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the subject matter of the present disclosure object requires more features than the features mentioned in the claims. Rather, the claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the terms “about”, “approximate”, or “substantially”. Unless otherwise stated, “about”, “approximate”, or “substantially” may indicate ±20% variation of the value it describes. Accordingly, in some embodiments, the numerical parameters used in the disclosure and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for preventing or treating ischemic stroke, comprising:
administering to a patient a drug containing a pharmaceutically effective amount of LY2922470, wherein a structural formula of the LY2922470 is:

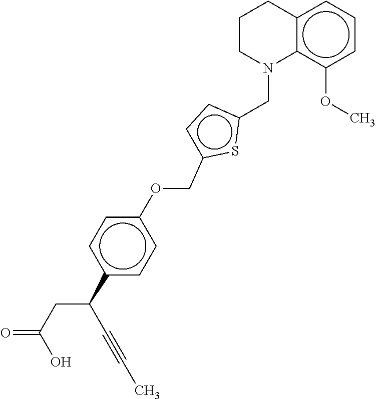

2. The method of claim 1, wherein the LY2922470 in the drug is in a form of a compound or a pharmaceutically acceptable salt thereof.

3. The method of claim 1, wherein the drug is in an oral or injectable dosage form, and the oral or injectable dosage form includes at least one of a powder, a tablet, a granule, a capsule, an oral liquid, an emulsion, and a suspension.

4. The method of claim 1, wherein the drug further contains an excipient.
